(19) United States
(12) Patent Application Publication
(10) Pub. No.: US 2025/0099172 A1
(43) Pub. Date: Mar. 27, 2025

(54) AXIALLY-ADJUSTABLE ELECTRODE TREATMENT SYSTEMS AND METHODS

(71) Applicant: Intuitive Surgical Operations, Inc., Sunnyvale, CA (US)

(72) Inventors: Jonathan COPE, Los Gatos, CA (US); Ryan C. ABBOTT, San Jose, CA (US); William CAMISA, Santa Clara, CA (US); David JOHNSON, Aptos, CA (US); Kollin TIERLING, Los Altos Hills, CA (US)

(73) Assignee: Intuitive Surgical Operations, Inc., Sunnyvale, CA (US)

(21) Appl. No.: 18/893,781

(22) Filed: Sep. 23, 2024

Related U.S. Application Data
(60) Provisional application No. 63/585,463, filed on Sep. 26, 2023.

(51) Int. Cl. A61B 18/14; A61B 18/00

(52) U.S. Cl. CPC A61B 18/1492; A61B 2018/00261; A61B 2018/00494; A61B 2018/00577; A61B 2018/00613; A61B 2018/1407; A61B 2018/1467

(57) ABSTRACT

Apparatuses, systems, and methods are disclosed for providing controlled delivery of energy treatment to a tissue site. The systems, apparatuses, and methods may include designs with features for efficiently deploying and adjusting electrodes at a tissue site for treatment and for facilitating retraction of the electrodes back into a housing after completion of treatment for removal. The systems, apparatuses, and methods may further include an expansion member for radially expanding the flexible substrate to aid in positioning the electrodes adjacent the tissue to facilitate treatment.

### CROSS-REFERENCE TO RELATED APPLICATION

This application claims priority benefit under 35 U.S.C. § 119 (e) of U.S. Provisional Patent Application No. 63/585,463 filed Sep. 26, 2023, the disclosure of which is hereby incorporated by reference in its entirety.

### TECHNICAL FIELD

The present disclosure relates generally to the field of tissue treatment. In particular, the disclosure relates to devices and methods with axially-adjustable electrodes for ablating cells to treat targeted tissues.

### BACKGROUND

Tissues in the body may be treated in surgical or medical procedures using resection and removal. While such procedures may be necessary to treat more extensive damage or resolve various medical conditions, minimally invasive procedures may be used as an alternative in some situations. Such procedures, where applicable, may be preferable to reduce overall patient risk, damage to surrounding tissue, recovery time, discomfort, and potentially harmful side effects as compared to more invasive procedures. In minimally invasive tissue ablation, targeted tissues may be treated inside the body (e.g., in situ) using procedures that do not involve resection or may require minimal resection. Some examples of minimally invasive tissue ablation techniques include electrolytic ablation, cryosurgery, chemical ablation (e.g., alcohol injection), thermal ablation (e.g., radiofrequency, microwave), and hydrothermal ablation. The primary aim of these ablation procedures is to destroy abnormal tissue in a targeted region and encourage the regrowth of healthy tissue.

Another minimally invasive treatment technique involves electroporation of targeted tissues by localized application of an electrical field to increase permeability of cell membranes, which may then allow drugs or other chemicals to be introduced into the cells for treatment. Electroporation may also be used in combination with electrolysis as a method of tissue ablation, by a process also known as electrolytic electroporation, electroporation-electrolysis, or E2.

Tissue ablation is a therapeutic procedure used to treat damage to various tissues, such as those of the gastrointestinal tract and other tissues. Damage to intestinal tissue may arise from several sources but is commonly associated with chronic metabolic disorders such as diabetes. For patients with diabetes, damage to gastrointestinal tissues (particularly the duodenum) may create issues with insulin resistance and/or impair the ability for the body to process glucose. To help manage the disorder, duodenal mucosal resurfacing (DMR) has been proposed to resurface the patient's intestinal lining in an effort to help regenerate healthy lining for improving nutrient absorption at the duodenum. Improved health of the intestinal lining may help correct absorption issues and yield a better therapeutic response to insulin for some patients, which in turn may allow patients to replace more aggressive insulin therapies (e.g., injections) with oral medications to help manage the condition.

Generally, DMR is a procedure that involves introducing a catheter (or other suitable medical instrument) into the duodenum, typically under endoscopic guidance, and ablating the inner lining of the duodenum. However, various ablation techniques and ablation instrument designs may not be suitable for endoluminal ablation or may be challenging to control ablation to desired depths via application from within the duodenum lumen.

The inventors have identified a need for an improved electrode treatment system for use with two or more electrodes to ablate cells for treating targeted tissues. In particular, an improved electrode treatment system for electroporation and/or electrolysis to ablate such cells. Some aspects of the improved electrode treatment system include axially-adjustable electrodes that may be arranged with a desired target spacing between the electrodes during, after, and/or prior to applying energy delivery for tissue treatment. The two or more electrodes can be two or more electrode arrays in the examples as described herein. Additional aspects and advantages of such systems will be apparent from the following detailed description of example embodiments, which proceed with reference to the accompanying drawings.

### SUMMARY

Various examples of electrode treatment systems for treating tissue are described herein. An example system may include a first flexible member with a first expandable support member coupled thereto and a second flexible member with a second expandable support member coupled thereto, wherein each of the first and second expandable support members is configured to expand from a stowed configuration to a deployed configuration. The system further includes a first electrode coupled to the first expandable support member and a second electrode coupled to the second expandable support member, wherein the electrodes are configured for collectively delivering energy to a tissue surface for treatment thereof, and wherein a position of the first expandable support member is adjustable independently of a position of the second expandable support member to adjust a target spacing between the first electrode and the second electrode for treatment of the tissue surface. In some example systems, the first electrode and the second electrode are electrode arrays each comprising a plurality of electrodes.

In some example systems, the first flexible member includes a hollow interior with a cavity extending therethrough and opening at a distal end portion of the first flexible member, wherein the second flexible member extends through the cavity and distally of the distal end portion of the first flexible member.

Some example systems may further include a sheath with a hollow interior with a cavity extending therethrough and opening at a distal end portion of the sheath, wherein both the first flexible member and the second flexible member each extend through the cavity and distally relative to the distal end portion of the sheath. In some example systems, the first expandable support member and the second expandable support member are in the stowed configuration within the sheath when the first flexible member and the second flexible member are positioned within the cavity of the sheath. In other example systems, the first flexible member and the second flexible member are movable along an axial direction through the cavity and distally of the sheath to expand the first expandable support member and the second expandable support member from the stowed configuration to the deployed configuration, wherein the first flexible member and the second flexible member are retractable along the axial direction into the cavity to retract the first expandable support member and the second expandable support member from the deployed configuration to the stowed configuration. In some example systems, the first expandable support member and the second expandable support member each spontaneously expand from the stowed configuration to the deployed configuration in response to the respective first flexible member and the second flexible member extending distally of the sheath. Some example systems may include one or more expansion members coupled to one or both of the first expandable support member and the second expandable support member, wherein the one or more expansion members are radially expandable to expand the one or both of the first expandable support member and the second expandable support member from the stowed configuration to the deployed configuration.

In some example systems, the first expandable support member and the second expandable support member each include a braided mesh frame. In some example systems, an exterior surface of the braided mesh frame includes a protective coating to reduce tissue trauma during delivery of the system to the tissue surface. In other example systems, the first expandable support member and the second expandable support member are each expandable balloons.

In some example systems, the first electrode and the second electrode are each ring-shaped structures disposed on a distal end portion of the first expandable support member and the second expandable support member, respectively. In other example systems, the first electrode and the second electrode are each point-contact electrodes disposed on a distal end portion of the first expandable support member and the second expandable support member, respectively.

Some example systems may include a locking mechanism operable to selectively lock the position of the first expandable support member relative to the position of the second expandable support member to maintain the target spacing between the first electrode and the second electrode during treatment of the tissue surface.

In some example systems, a frame extends between and couples the first expandable support member and the second expandable support member with one another, where the frame is adjustable to adjust the target spacing between the first electrode and the second electrode as desired. In some example systems, the frame, the first expandable support member, and the second expandable support member are each braided mesh frames. In other example systems, at least one expansion member is coupled to the frame, wherein the expansion member is radially expandable to expand the frame and adjust the target spacing of the first electrode and the second electrode.

Another example system for tissue treatment includes a delivery system having a first expandable support member and a second expandable support member, wherein the first expandable support member includes a first electrode coupled thereto and the second expandable support member includes a second electrode coupled thereto, wherein the first electrode and the second electrode are configured for delivering energy to a tissue surface for treatment thereof. The system further includes a controller in operable communication with the delivery system and configured to independently adjust a relative position of the first expandable support member and the second expandable support member based on a target energy level for application to the tissue surface to adjust a target spacing between the first electrode and the second electrode for treatment of the tissue surface, and to induce a voltage based on the target energy level and the target spacing of the first electrode and the second electrode to treat the tissue surface.

In some example systems, the first expandable support member is coupled to a first flexible member and the second expandable support member is coupled to a second flexible member, wherein the controller is operable to drive one or both of the first flexible member and the second flexible member relative to one another. In other example systems, the first flexible member includes a hollow interior with a cavity extending therethrough and opening at a distal end portion of the first flexible member, wherein the second flexible member is adjustable via the controller through the cavity and distally of a distal end portion of the first flexible member.

Some example systems further include a locking mechanism in operable communication with the controller, wherein the locking mechanism is operable to selectively lock the position of the first expandable support member relative to the position of the second expandable support member via the controller to maintain the target spacing between the first electrode and the second electrode during treatment of the tissue surface. In some example systems, the controller is further operable to adjust a rate of movement of the first expandable support member and the second expandable support member while inducing the voltage for continuous treatment of the tissue surface when the first electrode and the second electrode are locked in the target spacing.

Some example systems further include one or more expansion members coupled to one or both of the first expandable support member and the second expandable support member, wherein the one or more expansion members are radially expandable to expand the one or both of the first expandable support member and the second expandable support member and adjust the target spacing between the first electrode and the second electrode. In some example systems, the controller is in communication with the one or more expansion members, wherein the controller is operable to control an expansion rate of the one or more expansion members.

An example method for treating tissue via an electrode treatment system includes delivering a first electrode and a second electrode to a target tissue surface for treatment. The method further includes axially adjusting a position of one or both of the first electrode and the second electrode relative to each other to set a target spacing separating the first electrode from the second electrode at the target tissue surface. Thereafter, the first electrode and the second electrode are energized to deliver energy to the target tissue surface for treatment with the electrodes positioned at the target spacing. In some examples, the step of axially adjusting one or both of the first electrode and the second electrode may occur prior to energizing the electrodes or may occur during the energy delivery to the target tissue surface. In other examples, the first and second electrodes may be axially adjusted to retract the electrodes into a housing for removal upon completion of treatment. In other example methods, the first electrode is coupled to a first flexible member and the second electrode is coupled to a second flexible member, and the step of axially adjusting one or both of the first electrode and the second electrode further comprises axially adjusting one or both of the first flexible member and the second flexible member. In some examples, the first electrode and the second electrode are electrode arrays each comprising a plurality of electrodes.

Some example methods further include the step of determining a target energy level for application to the target tissue surface, wherein the step of axially adjusting one or both of the first electrode and the second electrode to set the target spacing is based on the determined target energy level.

In some example methods, the first electrode is coupled to a first expandable support member and the second electrode is coupled to a second expandable support member, and the method further includes expanding the first expandable support member and the second expandable support member to adjust a position of the respective first electrode and second electrode. In other example methods, the steps of expanding the first expandable support member and the second expandable support member precede the step of axially adjusting the position of one or both of the first electrode and the second electrode.

Some example methods further include the step of locking the first electrode and the second electrode at the target spacing prior to energizing the first electrode and the second electrode.

It should be understood that the foregoing summary provides various examples further described herein and is not intended to identify any key or critical aspects of the disclosed or claimed subject matter. Further, aspects of the example systems summarized above may be combined in any suitable manner without departing from the principles of the disclosed or claimed subject matter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are therefore not to be considered limiting in scope, the disclosure will be described with additional specificity and detail through use of the accompanying drawings, in which:

FIG. 1 is a schematic illustration of a system for electroporation and/or electrolysis in accordance with examples described herein.

FIG. 2 is a schematic illustration of a robotically-assisted manipulator system in accordance with examples described herein.

FIG. 3A is a schematic diagram of a medical instrument system in accordance with examples described herein.

FIG. 3B illustrates a distal portion of the medical instrument system of FIG. 3A with an extended example of an instrument in accordance with examples described herein.

FIG. 4 is a schematic of a medical procedure for inserting an electrode treatment system in accordance with examples described herein.

FIG. 5A is a schematic of the electrode treatment system of FIG. 4 for delivering electrodes to a target region for treatment in accordance with examples described herein.

FIG. 5B is a schematic of an electrode treatment system illustrating an axial repositioning of one electrode relative to another electrode in accordance with one example.

FIG. 6A illustrates an example of an electrode treatment system for delivering electrodes to a target region for treatment in accordance with examples described herein.

FIG. 6B illustrates the electrode treatment system of FIG. 6A with the electrodes in a stowed configuration within a sheath in accordance with one example.

FIG. 7 illustrates an electrode treatment system disposed within a lumen of a target tissue region for treatment in accordance with examples described herein.

FIG. 8 illustrates another example of an electrode treatment system for delivering electrodes to a target region for treatment in accordance with examples described herein.

FIG. 9 illustrates another example of an electrode treatment system for delivering electrodes to a target region for treatment in accordance with examples described herein.

FIG. 10 illustrates yet another example of an electrode treatment system for delivering electrodes to a target region for treatment in accordance with examples described herein.

### DETAILED DESCRIPTION

Certain details are set forth below to provide a sufficient understanding of embodiments of the disclosure. However, it will be clear to one skilled in the art that embodiments of the disclosure may be practiced without these particular details. Moreover, the particular embodiments of the present disclosure described herein are provided by way of example and should not be used to limit the scope of the claimed subject matter to these particular embodiments. In other instances, well-known materials, components, processes, controller components, software, and/or anatomy have not been described or shown in detail in order to avoid unnecessarily obscuring the embodiments.

This disclosure is directed to apparatuses and systems for providing controlled delivery of two or more electrodes (e.g., one or more pairs of electrodes) to a targeted tissue site for treatment (e.g., via electroporation and/or electrolysis). Generally, a method for minimally invasive regenerative surgery is disclosed which includes subjecting a target area in living tissue to ablation delivered via the two or more electrodes. In some examples, the ablation energy may be in the form of a combination of one or more electric fields and electrolysis delivered via the two or more electrodes. However, it should be appreciated that the example systems and methods described herein may be utilized for the deployment of electrodes using ablation modalities other than electroporation and/or electrolysis (e.g., radiofrequency ablation), or for purposes other than ablation, such as electrical stimulation or diagnostic methods.

During an example procedure utilizing electroporation and/or electrolysis, electrodes are brought into proximity and/or contact with the target tissue, and the electric fields are generated by applying voltages and/or currents between the one or more pairs of electrodes. The electric fields may be generated to have a magnitude to permeabilize cell membranes in a region where ablation is desired. The electric fields may be generated to produce products of electrolysis of a magnitude that, by themselves, do not cause damage to cells or the extracellular matrix located in and around the target treatment region. However, when sufficient products of electrolysis are generated in the region of permeabilized cells, cellular death occurs within the region of the applied electric field without damaging the extracellular matrix or scaffolding to aid in promoting tissue regrowth in the treated tissue region.

In some examples, after applying ablative energy to one target region, the electrodes may be moved to other tissue locations by advancing and/or retracting a delivery catheter and the electrodes carried by the delivery catheter as needed and repeating the process. In this manner, electroporation and/or electrolysis may be performed at multiple locations in a patient to cover a larger area of tissue. In some examples, multiple sets of electrodes may be positioned at multiple respective tissue sites such that electroporation and/or electrolysis may be performed at each site in parallel, reducing and/or eliminating a need to repeat the procedure as a catheter is moved through the patient.

In some embodiments of the present disclosure, the systems and methods disclosed herein may be used to treat tissue of the gastrointestinal tract. In other embodiments, any of a variety of tissue may be treated using the systems and methods described herein. Generally, tissue may be treated where tissue regeneration is desirable or where it is desirable to replace one type of cells with another. Examples include intestine, duodenum, stomach, bladder, uterus, endometrial lining, ovaries, colon, rectum, sinuses, ducts, ureters, prostate, skin, muscle, nerve, diaphragm, momentum, kidney, follicles, brain, lymphatic vessels, breast, esophagus, lung, liver, kidney, lymph nodes, lymph node basins and/or heart. Replacement of one type of tissue with another may be in fibrotic areas where it is desired to replace fibrotic cells with stem cells that can remodulate the area or when pancreatic islets are injected in part of the liver to generate new sources of insulin. Other tissue may be treated in other examples. The following provides additional details relating to example processes of electroporation and/or electrolysis for tissue treatment in accordance with some embodiments.

As noted above, electroporation may be performed to permeabilize the cell membranes of targeted cells. Reversible electroporation may be used in which the permeabilization may cease after the electric fields are removed. Cells may survive reversible electroporation with the pores within the membrane resealing and returning to homeostasis. In irreversible electroporation, the permeabilization of the cell membrane is permanent and leads to cell death. Typically for biological tissues, electric fields lower than about 1500 V/cm to about 200 V/cm are considered to produce reversible electroporation, while electric fields higher than about 1500 V/cm are considered to produce irreversible electroporation. Examples of systems and methods described herein may utilize reversible electroporation to avoid disadvantages of irreversible electroporation, which may include heating and thermal damage, complexity of providing such large electric fields, and muscle contractions which may result from the large electric fields. It is to be understood that, although systems and methods described herein may be designed to utilize reversible electroporation, there may occur localities or incidental areas where conditions are such that irreversible electroporation, or even thermal ablation, may occur in limited parts of the treated tissue.

In some treatment procedures, products of electrolysis may be applied to permeabilized cells to cause cell death of the permeabilized cells within the applied electrical field while leaving the extracellular matrix of the permeabilized cells intact. The extracellular matrix generally refers to a three-dimensional network of proteins and/or other molecules (e.g., collagen fibers, proteoglycans, and/or proteins such as fibronectin and/or laminin) which provide structure for cells and tissues and may additionally provide signaling for cell growth and development. Extracellular matrices may be used as scaffolds for tissue regeneration and/or engineering. For example, cells may be regenerated, grown, transplanted, or otherwise nurtured, on the extracellular matrix. By retaining the extracellular matrix in regions of otherwise ablated cells described herein, cell regeneration and/or tissue engineering may occur in the region of ablated cells. In some procedures, the extracellular matrix may be transplanted from a region of ablated cells to another region (which may be another region of ablated cells), to promote regeneration and/or tissue engineering in the transplanted region. In some cases, once the extracellular matrix is present, material may be injected into the extracellular matrix to enhance regrowth.

Example systems and methods described herein may utilize electrolysis products to cause cell death together with electroporation to permeabilize the cell membrane. The electrolysis products are sufficient to ablate (e.g., cause cell death) permeabilized cells in a relatively short time frame. However, the concentration of electrolysis products and exposure time are insufficient to cause cell death of non-permeabilized cells, thereby minimizing or avoiding the formation of scar tissue, fibrotic tissue, or ulceration in the treated region. Scar tissue and fibrosis is an indication that the extracellular matrix has been affected, and the ability for cells to regenerate and/or tissue engineering to occur in the ablated region may be inhibited. Accordingly, the electrolysis products are used to ablate permeabilized cells only, while leaving the extracellular matrix intact to promote tissue regrowth. Electrolysis products may include products toxic to cells (cytotoxic). Electrolysis preferentially utilizes one or more inert electrodes that do not participate in the process of electrolysis except as a source or sink of electrons or as catalysts. When participating non-inert electrodes are used in the process, they can generate metal ions that may cause systemic damage to the body, such as excess of iron or even metallic fragments.

Examples of systems disclosed herein include electrodes, a power supply, and a controller to apply energy for treating internal tissue. The energy applied may be electrolysis and/or electroporation in some examples, radiofrequency ablation in other examples, and other energy modalities in yet other examples. When the energy is electroporation with or without electrolysis, the controller controls a charge delivered to the electrodes to induce one or more electric fields. When electroporation and electrolysis are included, the electrodes are used to generate a current to produce electrolysis products and a voltage difference to produce an electric field that induces electroporation. The duration and magnitude of the charge applied determines the dose of the electrolytic products and the degree of the permeabilization of cells in the treatment site. Accordingly, a region of cell ablation may be determined by a region in which cells are exposed to the combination of permeabilization and to electrolysis products that cause ablation. The ablation, however, may leave the extracellular matrix intact in the region of ablated cells where the electrical field has been applied. The composition of the electrodes may be chosen in accordance with the desired products produced and electroporation effects.

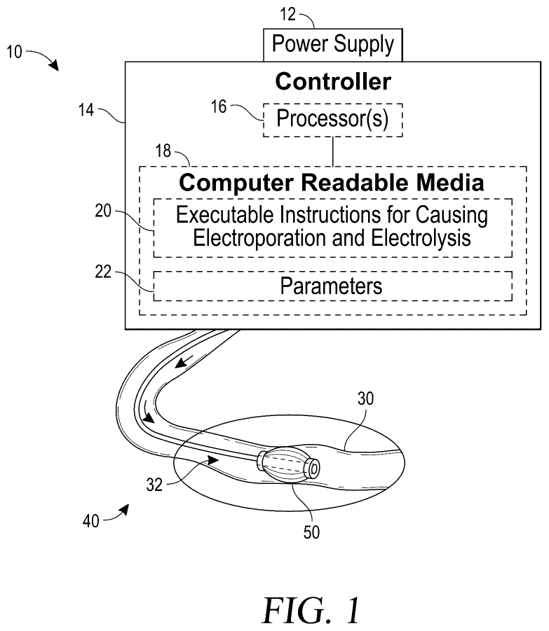

With reference to FIG. 1, the following provides a brief overview of an example system for delivering a medical instrument with electrodes to a targeted tissue site for treatment via electroporation and/or electrolysis. For clarity, the written description and the associated figures of various example embodiments may reference a catheter for use with the systems and methods described herein. However, it should be understood that other suitable medical instruments (e.g., instruments that may not be specifically classified as catheters) and other suitable energy modalities (e.g., radiofrequency ablation) may be used in conjunction with the disclosed systems and methods without departing from the principles of the disclosed subject matter.

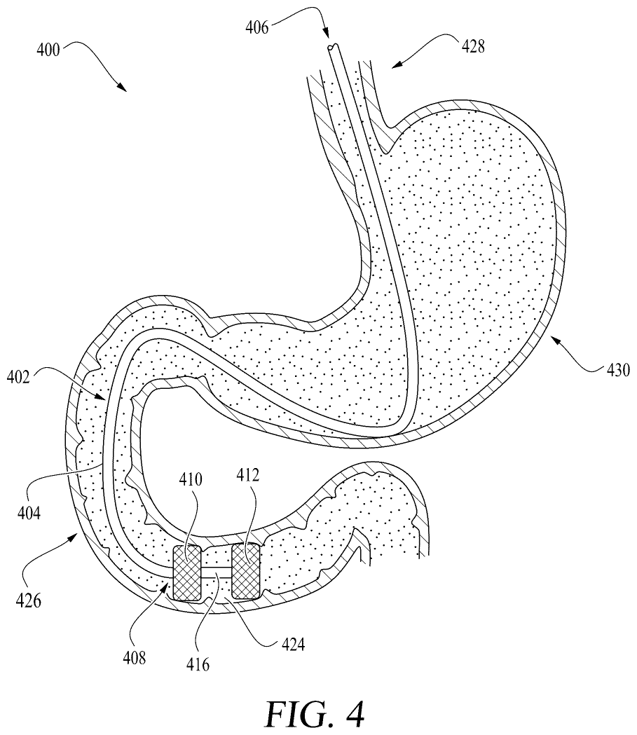

FIG. 1 is a schematic illustration of a system 10 arranged in accordance with example embodiments described herein. Generally, example systems described herein may include a delivery system and a controller. In the example of FIG. 1, the system 10 includes a controller 14 and a medical instrument system 40. Examples of other medical instrument systems, e.g., electrode treatment systems 400, 600, 700, 800, 900, 1000, which may be used with the system 10 (or other suitable systems described herein) are described in further detail below with respect to FIGS. 4-10. As further described below, the controller 14 may include a processor 16, computer readable media 18, and other computing system components, such as one or more input devices, output devices, sensors, and/or communication devices in some examples. Additional, fewer, and/or different components may be used in other examples. The computer readable media 18 includes executable instructions for causing electroporation and/or electrolysis 20 with the medical instrument system 40 and may include stored parameters 22 which may be used in the process for causing electroporation and/or electrolysis, such as electric field strengths, voltage and/or current levels, waveform shapes, exposure duration parameters, and any other suitable parameters. In the example of FIG. 1, the circle depicted around an end of the medical instrument system 40 may indicate a region of cells that may be permeabilized via an applied electric field.

The controller 14 may be implemented using a computing device. Examples of computing devices include controllers, microcontrollers, computers, servers, medical devices, smart phones, tablets, wearable devices, and the like. The computing device may be handheld and may have other uses as well. The controller 14 may include one or more processors, such as the processor 16. Any kind or number of processors may be present, including one or more central processing unit(s) (CPUs) or graphics processing unit(s) (GPUs) having any number of cores, controllers, microcontrollers, and/or custom circuity such as one or more application specific integrated circuits (ASICs) and/or field programmable gate arrays (FPGAs).

The controller 14 described herein may include computer readable media 18, such as memory. Any type or kind of memory may be present (e.g., read only memory (ROM), random access memory (RAM), solid state drive (SSD), secure digital card (SD card), and the like). While a single box is depicted as the computer readable media 18 in FIG. 1, any number of computer readable media 18 devices may be present. The computer readable media 18 may be in communication with (e.g., electrically connected to) the processor 16.

As noted above, the computer readable media 18 may store executable instructions for execution by the processor 16, such as executable instructions for causing electroporation and/or electrolysis 20 with the medical instrument system 40 and may utilize stored parameters 22 for the medical instrument system 40. In this manner, techniques for applying electroporation and/or electrolysis in tissue may be implemented herein wholly or partially in software.

The executable instructions 20 may include instructions to control a charge delivered to electrodes, such as electrodes 50 of the medical instrument system 40. Accordingly, the controller 14 may induce a voltage difference across the targeted tissue to generate an electric field that causes permeabilization of cells in an area of tissue targeted for treatment. The electrodes 50 are illustrated in a cavity or lumen 32 formed within tissue 30. Although the medical instrument system 40 is shown disposed within a cavity 32 of tissue 30, the medical instrument system 40 may be on the surface of the tissue 30, inside the tissue 30, and/or proximate to the tissue 30.

A controller, such as controller 14 of FIG. 1, may activate one or more selected electrodes 50 to provide an electric field. In some embodiments, the controller 14 may alternate or otherwise select a pattern of activated electrodes 50 (e.g., activating pairs of electrodes in sequence) to shape or deliver a particular electric field. In some examples, fluids or other substances may be injected into, brought into contact with, or otherwise placed in or around the tissue 30 to aid in shaping the electric field generated in the tissue 30. For example, conductive fluids may aid in shaping the field (e.g., by extending the field). In other examples, non-conductive fluids may aid in shaping the field (e.g., by attenuating the field). In some examples, non-conductive fluids or other substances may be injected or otherwise placed in tissue to protect areas where ablation is not desired. The electric field may not penetrate and/or not be carried through the non-conductive fluid, such that the field would not reach tissue where ablation is not desired, or at least be present in insufficient strength to cause permeabilization or other cellular change.

The controller 14 may also be used to induce a current through the tissue 30, such as between electrodes 50, to generate products of electrolysis. The products of electrolysis may cause ablation of the permeabilized cells but are preferably insufficient to destroy the extracellular matrix in the region of the permeabilized cells. The remaining intact extracellular matrix may allow for regeneration of the tissue and tissue engineering as described previously.

In some embodiments, one or more of the electrodes 50 used to apply electroporation may also be used to generate products of electrolysis (in other words, some or all of the electrodes may be used for both electroporation and electrolysis). In other embodiments, a first subset of electrodes 50 used to apply electroporation may be different from a second subset of electrodes 50 used to generate products of electrolysis.

In some embodiments, the power supply 12 is integrated with the controller 14. The power supply 12 may be implemented using any suitable power source, such as one or more AC power sources, DC power sources, batteries, and/or waveform generators. The power supply 12 may supply power to the electrodes 50 to generate a voltage and/or current and, therefore, an electric field and/or electrolysis products in the tissue 30. In some examples, the power supply 12 may be implemented using a signal generator, such as an exponential decay wave generator (by way of example, a Harvard Apparatus BTX 630), however, this disclosure is not limited thereto or thereby.

The controller 14 may control the timing, strength, and duration of electric fields and/or electrolysis products provided via the medical instrument system 40. The controller 14 may, for example, be programmed to provide an electronic signal to the medical instrument system 40, via the power supply 12, where the electronic signal may be indicative of a dose of treatment, for example, a dose of electrolysis products and/or permeability level of cell. The electronic signal may control the timing and magnitude of the generated electric field, which may allow a user to customize treatment of the tissue 30 as desired. In some embodiments, the controller 14 may include such a program, or include one or more processing devices (e.g., processors), coupled to the computer readable media 18 encoded with executable instructions for electrolysis and permeabilization 20. Although shown as a separate component coupled to the medical instrument system 40 in FIG. 1, in some embodiments, the controller 14 may be integrated as part of the medical instrument system 40. In other embodiments, the controller 14 may include programmable circuitry coupled to the medical instrument system 40 via a wired or wireless connection.

As noted previously, the system 10 may include any suitable parameters 22 for controlling various aspects of the electroporation and/or electrolysis processes, such as electric field strengths, voltage levels, current levels, waveform shapes, exposure duration parameters, and any other suitable parameters. The parameters 22 may be stored in computer readable media 18 or in other suitable databases in communication with the controller 14. In some embodiments, the controller 14 may be used to calculate the parameters 22, or the controller 14 may be in communication with another system operable to calculate the parameters 22.

In some example embodiments, the parameters 22 for a specific treatment protocol may be determined based on measurements taken in the tissue of interest or from a different sample of similar tissue. For example, measurements may be taken at various voltage levels with particular electrode configurations, and a target voltage level, current, pulse pattern, time constant, and other factors may be identified which cause reversible electroporation and the delivery of electrolysis products to result in cell death of the permeabilized cells as desired.

In some embodiments, examples of parameters 22 which may be used include a delivery of between 1 and 50 voltage pulses between 50 V and 1000 V. Those pulses may be delivered in a system having a capacitance between 50 μF and 1000 μF, and a resistance of between 15-20 ohms. A quantity of electrolysis products generated may be related to the delivered charge in Coulombs. There are several ways to calculate the delivered charge. For example, the stored electric charge in a capacitor, Q (in coulombs, abbreviated C) is equal to the product of the capacitance C (in Farads, abbreviated F) of the capacitor, and the voltage V (in volts, abbreviated V) across its terminals. That is, Q=C·V. Also, current I (in amps, abbreviated A) multiplied by time t=charge. I*t=Q. By defining the capacitance and the voltage across the capacitance, the charge may be defined, and accordingly the electrolysis performance determined. When a capacitor is discharged, it generates current and the current multiplied by time must be equal to the charge in the capacitor. When a capacitor is being discharged the current is not constant-it decays exponentially. Therefore, the time measure is given as the exponential decay time constant. The capacitance which controls the time constant is generally obtained from capacitors incorporated in the power supply, such as power supply 12 of FIG. 1.

In some embodiments, the time constant (e.g., exponential decay time constant of the capacitive discharge) may range from 50 microsecond (μs) to 3 ms. Generally, the lower limit of the time constant is related to a time sufficient to ensure electrolytic species (e.g., products of electrolysis) permeate the targeted area of permeabilized cells. The upper limit of the time constant is generally related to the production of electrolytic species (e.g., products of electrolysis) that can cause ablation on their own. Accordingly, electrolysis is generally targeted for an amount of time sufficient to allow diffusion of electrolysis products through a region of permeabilized cells. However, the amount of time electrolysis is provided should be limited to ensure the process does not result in ablation of non-permeabilized cells or otherwise damages the extracellular matrix in the region of the ablated cells.

In some embodiments, the generated electric field ranges between 100 V/cm and 3500 V/cm. In other embodiments, the electric field may be between 100 V/cm and 1500 V/m or between 200 V/cm and 850 V/cm. In still other embodiments, the electric field may be less than 1400 V/cm in some examples, less than 1300 V/cm in some examples, less than 1000 V/cm in some examples, less than 800 V/cm in some examples, or less than 600 V/cm in some examples.

The system 10 may further include one or more sensors (not shown) for measurement of pH, temperature, electric field strength, tissue electrical resistivity or impedance, and/or other suitable properties of the tissue 30 for optimizing treatment. For example, in one embodiment, a pH sensor may be incorporated with the system 10. The pH sensor may be arranged in any one of several configurations, such as coupled to the medical instrument system 40 adjacent the electrodes 50 to detect a pH value near the electrodes 50. In another embodiment, a pH sensor may be provided at an outer edge of a targeted region of tissue. In either configuration, the pH sensor may be in communication with the controller 14, where the controller 14 may utilize one or more received pH values as an indication of tissue ablation and/or to monitor the occurrence of tissue damage based on detected pH levels at the treatment site and surrounding regions of tissue. In some embodiments, the controller 14 may adjust the voltage, current, and/or electric field applied to the tissue responsive to the detected pH levels. For example, if pH values for tissue located outside the treatment site are at or exceed a threshold for tissue damage, the controller 14 may reduce a magnitude of electric field, a duration between pulses, or cease application of the electric field. Similarly, if pH values for tissue within a targeted region are at or exceed a threshold for tissue ablation, the controller 14 may cease application of current through electrodes immediately and/or after a desired elapsed electrolysis time to cease the electrolysis process.

In some embodiments, a resistivity meter may be used to determine a resistance of the target tissue. For example, the controller 14 and/or power supply 12 of FIG. 1 may provide an impedance measurement. The impedance measurement may determine a resistivity of the tissue 30 contacted by electrodes 50 of the system 10. For example, the controller 14 and/or power supply 12 may provide a nominal amount of current, such as DC current, through the tissue 30 and receive a resistivity measurement and/or calculate resistivity of the tissue 30. In some examples, an applied voltage, current, and/or electric field may be selected, determined, and/or allowed based on a measured resistance of the tissue 30. In some examples, a number of pulses of applied voltage may be selected, determined, and/or otherwise used based on a measured resistance of the tissue.

In some embodiments, a sensor, such as a Gauss meter and/or Tesla meter, for detecting and/or determining electric field strength may also be used. In such embodiments, the sensor may be in operable communication with the controller 14 for ensuring that the electric field strength remains at a target level for a desired treatment protocol.

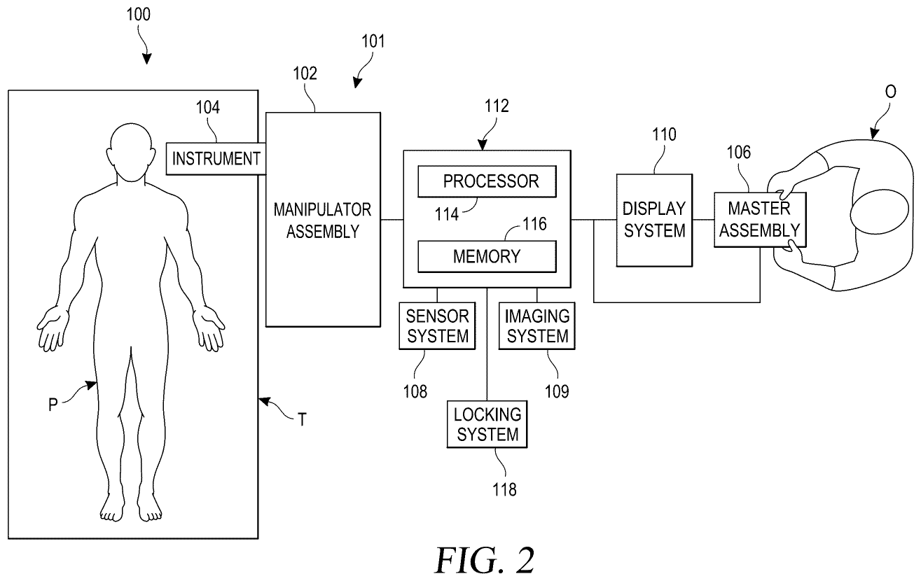

In some embodiments, the ablation instrument for delivering electrolytic electroporation (e.g., medical instrument system 40 of FIG. 1) may be delivered via a computer-assisted, teleoperational manipulator system, sometimes referred to as a robotically assisted system or a robotic system. The manipulator system comprises one or more manipulators that can be operated with the assistance of an electronic controller (e.g., computer) to move and control functions of one or more instruments when coupled to the manipulators. FIG. 2 illustrates one example embodiment of a robotically-assisted manipulator system 100 for use with the systems and methods described herein. The manipulator system can be used, for example, in surgical, diagnostic, therapeutic, biopsy, or non-medical procedures.

With reference to FIG. 2, a robotically-assisted manipulator system 100 may include one or more manipulator assemblies 102 for operating one or more medical instrument systems 104 in performing various procedures on a patient P positioned on a table T in a medical environment 101. For example, the manipulator assembly 102 may drive medical instrument or end effector motion, may apply treatment to target tissue, and/or may manipulate control members. The manipulator assembly 102 may be teleoperated, non-teleoperated, or a hybrid teleoperated and non-teleoperated assembly with select degrees of freedom of motion that can be motorized and/or teleoperated and select degrees of freedom of motion that may be non-motorized and/or non-teleoperated. An operator input system 106, which may be inside or outside of the medical environment 101, generally includes one or more control devices for controlling manipulator assembly 102. The manipulator assembly 102 supports a medical instrument system 104 and may optionally include a plurality of actuators or motors that drive inputs on the medical instrument system 104 in response to commands from a control system 112. The actuators may optionally include drive systems that when coupled to the medical instrument system 104 advance the medical instrument system 104 into a naturally or surgically created anatomic orifice. Other drive systems can move the distal end of the medical instrument system 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 102 may support various other systems for irrigation, treatment, or other purposes. Such systems may include fluid systems (including, for example, reservoirs, heating/cooling elements, pumps, and valves), generators, lasers, interrogators, and ablation components.

The robotically-assisted manipulator system 100 also includes a display system 110 for displaying an image or representation of the surgical site and the medical instrument system 104 generated by an imaging system 109 which may include an imaging system, such as an endoscopic imaging system. The display system 110 and the operator input system 106 may be oriented so an operator O can control the medical instrument system 104 and the operator input system 106 with the perception of telepresence. A graphical user interface may be displayable on the display system 110 and/or a display system of an independent planning workstation.

In some examples, the endoscopic imaging system components of the imaging system 109 may be integrally or removably coupled to the medical instrument system 104. However, in some examples, a separate imaging device, such as an endoscope, attached to a separate manipulator assembly may be used with the medical instrument system 104 to image the surgical site. The endoscopic imaging system 109 may be implemented as hardware, firmware, software, or a combination thereof which interact with or are otherwise executed by one or more computer processors, which may include the processors of the control system 112.

The robotically-assisted manipulator system 100 may also include a sensor system 108. The sensor system 108 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 104. The sensor system 108 may also include temperature, pressure, force, or contact sensors or the like.

The robotically-assisted manipulator system 100 may also include a control system 112. The control system 112 includes at least one memory 116 and at least one computer processor 114 for effecting control between the medical instrument system 104, the operator input system 106, the sensor system 108, and the display system 110. The control system 112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

The control system 112 may optionally further include a virtual visualization system to provide navigation assistance to operator O when controlling the medical instrument system 104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 112 may use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

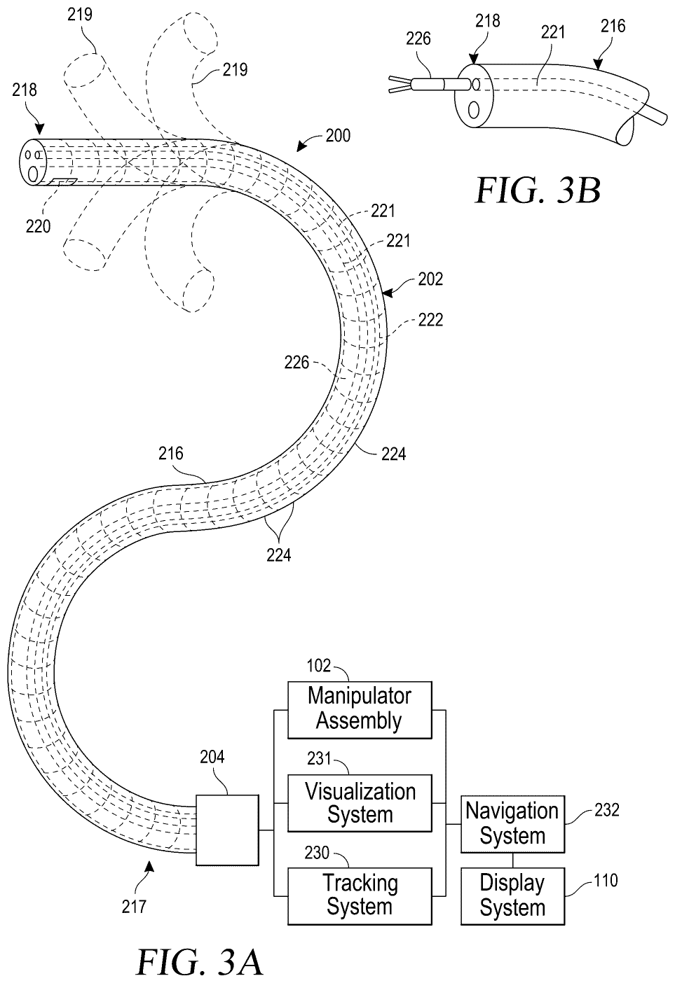

FIG. 3A illustrates a medical instrument system 200 according to some example embodiments. The medical instrument system 200 may be used in an image-guided medical procedure. In some embodiments, the medical instrument system 200 may be used for non-teleoperational exploratory procedures or in procedures involving traditional manually operated medical instruments, such as endoscopy. In other embodiments, the medical instrument system 200 is interchangeable with, or a variation of, the medical instrument system 104 of FIG. 2.

The medical instrument system 200 includes an elongate flexible device 202, such as a flexible catheter, endoscope (e.g., gastroscope, bronchoscope), or other suitable device coupled to a drive unit 204. The elongate flexible device 202 includes a flexible body 216 having a proximal end portion 217 and a distal end portion 218, including a tip portion. In some embodiments, the flexible body 216 has an outer diameter of approximately 14-20 mm. Other embodiments of the flexible body 216 may have larger or smaller outer diameters. The flexible body 216 may have an appropriate length to reach certain portions of the anatomy, such as the lungs, sinuses, throat, or the upper or lower gastrointestinal region, when the flexible body 216 is inserted into a patient's oral or nasal cavity.

The medical instrument system 200 optionally includes a tracking system 230 for determining the position, orientation, speed, velocity, pose, and/or shape of the distal end portion 218 and/or of one or more segments 224 along the flexible body 216 using one or more sensors and/or imaging devices. The entire length of the flexible body 216, between the distal end portion 218 and the proximal end portion 217, can be effectively divided into segments 224. The tracking system 230 may optionally be implemented as hardware, firmware, software or a combination thereof which interact with or are otherwise executed by one or more computer processors, which may include the processors of the control system 112 in FIG. 2.

The tracking system 230 may optionally track the distal end portion 218 and/or one or more of the segments 224 using a shape sensor 222. In some embodiments, the tracking system 230 may optionally and/or additionally track the distal end portion 218 using a position sensor system 220, such as an electromagnetic (EM) sensor system. In some examples, the position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

The flexible body 216 includes one or more channels (e.g., passageways) sized and shaped to receive one or more medical instruments 226. In some embodiments, the flexible body 216 includes two channels 221 for separate instruments 226, however, a different number of channels 221 may be provided. FIG. 3B is a simplified diagram of an end portion of the flexible body 216 with the medical instrument 226 extended outwardly therefrom according to some embodiments. In some embodiments, the medical instrument 226 may be used for procedures and aspects of procedures, such as surgery, biopsy, ablation, mapping, imaging, illumination, irrigation, or suction. The medical instrument 226 may be deployed through the channel 221 of the flexible body 216 and used at a target location within the anatomy. The medical instrument 226 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, expandable ablation members, transducers, sensors, imaging devices and/or the like. The medical instrument 226 may be advanced from the opening of channel 221 to perform the procedure (e.g., electroporation and/or electrolysis in the present disclosure) and then retracted back into the channel 221 when the procedure is complete. The medical instrument 226 may be removed from the proximal end 217 of the flexible body 216 or from another optional instrument port (not shown) along the flexible body 216. The medical instrument 226 may be used with an image capture device (e.g., an endoscopic camera) also within the elongate flexible device 202. Alternatively, the medical instrument 226 can itself be the image capture device.

The medical instrument 226 may additionally house cables, linkages, or other actuation controls (not shown) that extend between its proximal and distal end portions 217, 218 to controllably bend the distal end portion 218 of the medical instrument 226. The flexible body 216 may also house cables, linkages, or other steering controls (not shown) that extend between the drive unit 204 and the distal end portion 218 to controllably bend the distal end portion 218 as shown, for example, by the broken dashed line depictions 219 of the distal end portion 218. In some examples, at least four cables are used to provide independent “up-down” steering to control a pitch motion of the distal end portion 218 and “left-right” steering to control a yaw motion of the distal end portion 218. In embodiments in which the medical instrument system 200 is actuated by a robotically-assisted assembly, the drive unit 204 can include drive inputs that removably couple to and receive power from drive elements, such as actuators, of the teleoperational assembly. In some embodiments, the medical instrument system 200 can include gripping features, manual actuators, or other components for manually controlling the motion of the medical instrument system 200. The information from the tracking system 230 can be sent to a navigation system 232 where it is combined with information from the visualization system 231 and/or the preoperatively obtained models to provide the physician or other operator with real-time position information.

In some embodiments, the medical instrument system 200 may be guided manually or via the robotically-assisted manipulator system 100 to deliver the medical instrument 226 to a target tissue site for treatment. In some procedures, the selection between a manual or robotic delivery and the approach may be determined based on the medical application. For example, in some gastrointestinal applications, the medical instrument system 200 (or standalone medical instrument 226) may be delivered via a manual or robotic delivery device endoluminally through a trans-oral or trans-anal approach. A trans-abdominal approach with integrated monopolar or bipolar instrumentation, drop in probes or via catheters may also be used. For urological applications, the medical instrument system 200 (or standalone medical instrument 226) may be delivered via a manual or robotic delivery device endoluminally through a trans-urethral approach, trans-perineal, pre-peritoneal or trans-abdominally with integrated bipolar instrumentation, drop in probes or via catheters. Similarly, for gynecological applications, the medical instrument system 200 (or standalone medical instrument 226) may be delivered via a manual or robotic delivery device endoluminally through a trans-vaginal approach, trans-perineal, or trans-abdominally with integrated bipolar instrumentation, drop in probes or via catheters. For hepatobiliary applications, the medical instrument system 200 (or standalone medical instrument 226) may be delivered via a manual or robotic delivery device endoluminally through a trans-oral approach to reach the ampulla or to go externally into the liver via a trans-gastrointestinal wall route or trans-abdominally with integrated bipolar instrumentation, drop in probes or via catheters. For neurovascular applications, the medical instrument system 200 (or standalone medical instrument 226) may be delivered via a manual or robotic delivery device through an endovascular approach or through a keyhole craniotomy with integrated bipolar instrumentation, drop in probes or via catheters. For cardiac applications, the medical instrument system 200 (or standalone medical instrument 226) may be delivered via a manual or robotic delivery device through an endovascular approach or trans-thoracic with integrated bipolar instrumentation, drop in probes or via catheters.

With collective reference to FIGS. 4-10, the following provides additional details of various embodiments and features relating to electrode treatment system designs and various components thereof that may be used together with the system 10, the robotically-assisted manipulator system 100, and/or the medical instrument system 200 described previously with reference to FIGS. 1-3B. Generally, the electrode treatment systems described with reference to FIGS. 4-10 are designed to deliver electrodes for treatment of a target tissue site, where the electrodes may be independently controllable to adjust a spacing of the electrodes as desired for treatment of a target tissue surface while maintaining an overall compact profile of the electrode treatment system to facilitate delivery to the target tissue site. Other features and advantages of the electrode treatment system designs are further described below with reference to the figures.

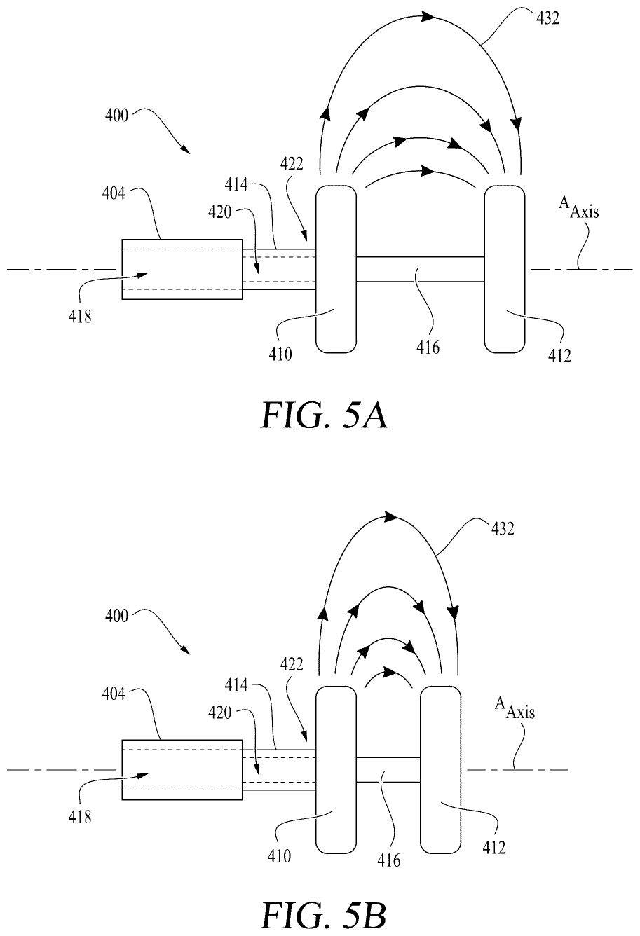

FIGS. 4-5B collectively illustrate an electrode treatment system 400 operable for delivering electrodes 410, 412 to a target tissue site 424 in accordance with an example embodiment. In some example embodiments of the present disclosure, the electrode treatment system 400 may be used to provide electrolysis and/or electroporation treatment to the target tissue site 424, such as for duodenal mucosal resurfacing in the gastrointestinal tract. In other embodiments, the electrode treatment system 400 may be used to treat other target tissue using other treatment processes.

With general reference to FIGS. 4-5B, the electrode treatment system 400 includes an elongate flexible device 402, such as a flexible catheter, an endoscope (e.g., duodenoscope, gastroscope, bronchoscope) or other suitable member, with a body 404 having a proximal portion 406 and a distal portion 408. In some embodiments, the body 404 has an outer diameter which may depend on the target anatomy for treatment. In some examples, the outer diameter may range between 2 mm and 12 mm. The body 404 may have an appropriate length to reach certain portions of the anatomy, such as the lungs, sinuses, throat, or the upper or lower gastrointestinal region, when the elongate flexible device 402 is inserted into a patient's oral or nasal cavity (or otherwise introduced, such as via a surgical incision). In some embodiments, the length of the elongate flexible device 402 may range between 85 cm and 300 cm (or another suitable length) to reach a desired anatomical tissue region for treatment. It should be understood that in other embodiments, the elongate flexible device 402 and its components may have any suitable dimensions that may depend on the target tissue region, surrounding anatomical structures, or other suitable factors.

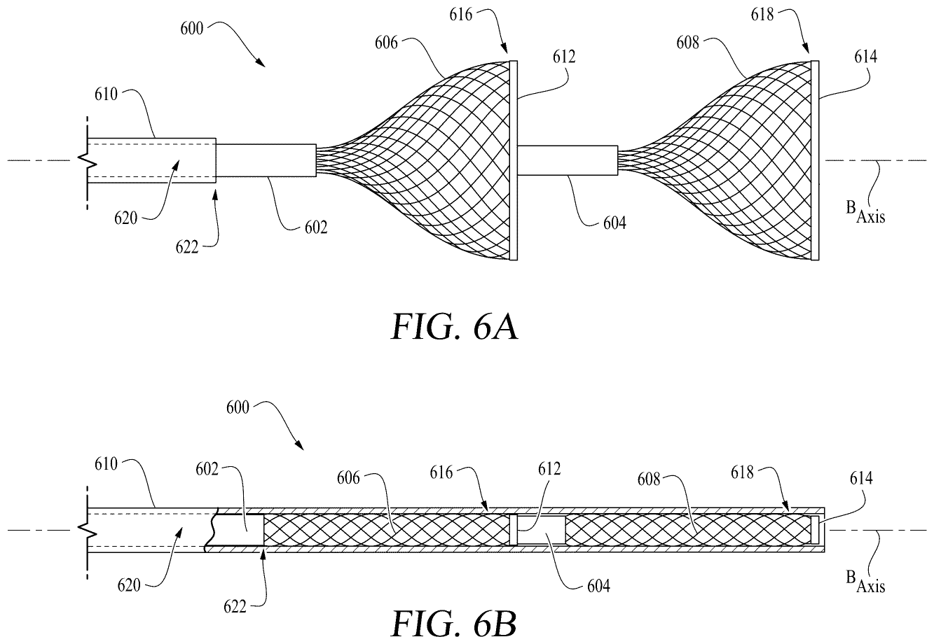

With reference to FIG. 5A, the electrode treatment system 400 further includes a first electrode 410 and a second electrode 412 laterally or axially spaced apart from one another (e.g., along a longitudinal axis A), where the electrodes 410, 412 are at least suitable for providing one or both of electrolysis and electroporation to the targeted tissue in a similar fashion as described previously. The electrodes 410, 412 may take any suitable configuration operable for generating an electric field (e.g., as illustrated by lines 432 in FIGS. 5A, 5B). For example, in one embodiment, the electrodes 410, 412 may comprise a plurality of metal traces deposited on expandable support members. Some example configurations for the electrodes 410, 412 and expandable support members of the electrode treatment system 400 are provided with particular reference to FIGS. 6A-10 as further discussed below. The metal traces may include any suitable conductive material such as gold, copper, stainless steel, titanium, graphene, graphite, and the like. Generally, electrode materials may be selected such that the electrode material does not actively participate in electrolysis products and/or leave material residue. In some embodiments, the electrode material may be chosen to minimize transferring ions from the electrode material to the target tissue. Titanium and gold may have some degree of participation in the electrolysis process, however, they may not generate a toxic residue and may be used in some examples. In other embodiments, the electrodes 410, 412 may comprise other suitable shapes and configurations, including circular, square, rectangular, or other shapes for the electrodes. Interdigitated electrodes may also be used. The electrodes 410, 412 and other electrodes included in the electrode treatment systems described herein may each be part of arrays of two or more electrodes.

Generally, the electrodes used in the example configurations described herein are arranged in a bipolar configuration. Generally, in a bipolar configuration, current may travel from one electrode of a pair of electrodes to another electrode of the pair of electrodes. The electrodes in the pair may accordingly be said to be of opposite polarity. In some configurations, multiple pairs of electrodes may be used, with the current passing through each pair of electrodes.

In other examples, the electrodes may be arranged in a monopolar configuration or may be arranged in a combination of bipolar and monopolar configurations. A monopolar configuration may include an active electrode (e.g., an electrode on or in the surgical field) and a return electrode. The return electrode may be placed outside the surgical field but in contact with the patient in some examples (e.g., using a pad having an electrode). In this manner, one polarity (e.g., the polarity of the active electrode) is in the surgical field. Accordingly, a monopolar configuration of electrodes may include a pair of electrodes-with one energized and one serving as a return. The return electrode may not be provided on a same device as the energized electrode. For example, the return electrode may be provided on a pad placed proximate the tissue. In a monopolar configuration, multiple active electrodes (e.g., two or more electrodes 410, 412) may be present and may pass current through a shared return electrode in some examples, or through respective return electrodes in other examples.

The electrode treatment system 400 further includes suitable wiring, circuitry, and other electronic components (not shown) for providing electrical signals, such as voltages and/or currents, to the electrodes 410, 412. In one embodiment, the wiring may run down the length of the elongate flexible device 402 to the controller 14 and/or power supply 12 (see FIG. 1) or to the control system 112 (see FIG. 2) which may be outside the body. In other embodiments, other suitable wiring configurations may be used to power the electrodes 410, 412.

With particular reference to FIG. 5A, the following describes additional details relating to one example arrangement of the electrodes 410, 412 with respect to the elongate flexible device 402. With reference to FIG. 5A, the electrode treatment system 400 may include a first flexible member 414 supporting the electrode 410 and a second flexible member 416 supporting the second electrode 412, where both flexible members 414, 416 are disposed within or extend through a cavity 418 of the elongate flexible device 402. The flexible members 414, 416 may be any suitable elements capable of being housed within and driven relative to the elongate flexible device 402. In some example systems, the flexible members 414, 416 may include catheters, guidewires, shafts, or other suitable drive elements capable of being driven manually or via a control system (e.g., control system 112 of FIG. 2).

In one example configuration, the first flexible member 414 further includes a hollow interior with a cavity 420 extending therethrough and opening at a distal end portion 422 of the first flexible member 414. In some embodiments, the second flexible member 416 extends through the cavity 420 of the first flexible member 414 and distally of the distal end portion 422 of the first flexible member 414 (e.g., when the electrode treatment system 400 is in the deployed or expanded configuration). As described, the first flexible member 414 and the second flexible member 416 are arranged in a generally telescoping configuration where the second flexible member 416 is axially moveable within the cavity 420 of the first flexible member 414. In this configuration, the first flexible member 414 and the second flexible member 416 are independently adjustable relative to one another to adjust a spacing between the first electrode 410 and the second electrode 412 for treatment of the tissue surface as further described below. The flexible members 414, 416 may be adjustable via the control system 112 (see FIG. 2) or a controller 14 (see FIG. 1), or may be manually adjustable by an operator O, or otherwise adjustable via a combination of robotic and/or manual adjustment. Additional details of other example arrangements for electrode treatment systems are provided further below with reference to FIGS. 6A-10.

With general reference to FIGS. 4-5B, the following describes an example delivery procedure of the electrode treatment system 400 to a target tissue site 424 for treatment in accordance with an example embodiment. In one example, the elongate flexible device (e.g., endoscope) may be delivered manually or via a computer-assisted teleoperational manipulator system (see FIG. 2) in a trans-oral manner to the duodenum 426 for ablation of mucosal lining (or other tissue site). For this procedure, delivery may include entry through the patient's esophagus 428, navigation through the stomach 430, and finally into the duodenum 426 for treatment. In other examples, delivery may be made via other routes, including via openings created by surgical incisions, and may include the use of other delivery systems.

With the electrode treatment system 400 in position at the duodenum 426, the first flexible member 414 and the second flexible member 416 are advanced (either independently or substantially simultaneously) axially through the cavity 418 of the elongate flexible device 402 (or through a cavity of another suitable housing if not using an elongate flexible device 402) and distally through the distal end portion 408 thereof. Upon deployment of the flexible members 414, 416 distally of the elongate flexible device 402, the electrodes 410, 412 expand (e.g., passively or actively as described in more detail below with reference to FIGS. 6A and 6B) within the duodenum 426 until the electrodes 410, 412 are in proper position relative to the duodenum 426. In some embodiments, the electrodes 410, 412 may be in contact with the duodenum 426, while in other embodiments, the electrodes 410, 412 may be adjacent, but not in contact, with the duodenum 426.

An axial or lateral position of one or both of the electrodes 410, 412 may be adjusted to ensure a desired target spacing is achieved and maintained between the electrodes 410, 412 for treatment (e.g., prior to, during, and/or after energy delivery or expansion of the electrodes). For example, as illustrated in FIG. 5B, in one embodiment, one or both of the flexible members 414, 416 may be moved relative to the other via the controller 14, the control system 112, or manually to position the electrodes 410, 412 at the desired target spacing. In FIG. 5B, the second electrode 412 has been retracted to move the second electrode 412 closer to the first electrode 410 (as compared to a position illustrated in FIG. 5A). In other embodiments, the electrode(s) may be moved farther apart. In this configuration, the electric field (as illustrated by lines 432 in FIG. 5B) between the electrodes 410, 412 is more compact as compared to the arrangement of the electrodes 410, 412 of FIG. 5A. In some embodiments, an imaging system, sensor system, or other suitable visualization system (not shown) of the electrode treatment system 400 may be used to guide the adjustment and verify that the electrodes 410, 412 are arranged in a desired position at the target tissue site 424. Once the electrodes 410, 412 are in position, the electrodes 410, 412 may be energized to ablate target cells within the duodenum 426 as described previously. In some embodiments, the electrode treatment system 400 and other electrode treatment systems described herein may be manually actuated by a user. In other embodiments, the electrode treatment systems may be coupled to an external device for actuation such as a robotically-assisted manipulator system (e.g., robotically-assisted manipulator system 100). In yet other embodiments, manual and robotically-assisted actuation may be provided.

The electrode treatment systems described herein may be coupled to an external device to provide actuation, control, and/or electrical power to the electrode treatment system. For example, the electrode treatment system 400 and the other electrode treatment systems described herein may be actuated by a robotically-assisted manipulator system (e.g., manipulator assembly 102). The electrode treatment system 400 (e.g., elongate flexible device 402, first flexible member 414, second flexible member 416, and/or expandable support members as described in more detail with respect to electrode treatment system 600) may include or be coupled to a drive unit (e.g., drive unit 204) having one or more drive inputs (e.g., at proximal portion 406) that removably couple to and receive power from drives elements, such as actuators or motors, of the manipulator assembly 102. When the electrode treatment system 400 is coupled to the manipulator assembly, the drive inputs of the electrode treatment system 400 may be coupled to drive outputs of the manipulator assembly that are driven by the drive elements of the manipulator assembly. In such embodiments, the drive inputs of the electrode treatment system 400 may be coupled to the distal end portion of the electrode treatment system 400 via one or more actuation drive members or elements (e.g., drive shafts, actuation cables, actuation rods, tension members, and the like) to perform actions such as advancing, retracting, or articulating the distal end of the electrode treatment system 400. Similarly, the drive inputs of the electrode treatment system 400 may be coupled to the first and second flexible members via one or more actuation drive members or elements to perform actions such as advancing, retracting, or articulating one or both of the flexible support members to adjust the relative axial spacing between the electrodes coupled thereto. Additionally, the drive inputs of the electrode treatment system 400 may be coupled to the electrodes or expandable support members to perform actions such as expansion, deployment, and/or retraction of the electrodes and/or expandable support members described herein between a deployed position for treatment and a stowed position for retraction. In addition, the drive unit 204 may be coupled to a controller (e.g., controller 14) and/or power supply (e.g., power supply 12) to provide electrical power to electrodes of electrode treatment system 400 as described above. The drive unit 204 may be electrically coupled to the electrodes via one or more electrical cables running along or through the electrode treatment system 400. In alternative embodiments, the controller and power supply may provide power to the electrical cables without coupling to the drive unit (e.g., the controller and power supply may be independent of the drive unit).

With general reference to FIGS. 6A-10, the following section provides additional details for other electrode treatment system embodiments that may be used together with the systems 10, 100, 200 described previously. FIG. 6A illustrates an example of an electrode treatment system 600 for delivering electrodes 612, 614 to a target region for treatment in accordance with examples described herein. Generally, the electrode treatment system 600 may have the same or substantially similar features and operate in a substantially similar manner as the electrode treatment system 400 described previously with reference to FIGS. 4-5B. Accordingly, certain details of the electrode treatment system 600 may not be further described in detail below to avoid repetition and/or obscuring more pertinent details of the embodiment. It should be understood that the description of corresponding components and their related functionality as described with reference to electrode treatment system 400 may apply, in whole or in part, equally to the electrode treatment system 600 unless otherwise described below.

With reference to FIG. 6A, the electrode treatment system 600 includes a first flexible member 602 and a second flexible member 604. In some embodiments, the flexible members 602, 604 may include catheters, guidewires, shafts, or other suitable drive elements capable of being driven manually or via a control system (e.g., control system 112 of FIG. 2). The first flexible member 602 includes a first expandable support member 606 coupled thereto and the second flexible member 604 includes a second expandable support member 608 coupled thereto. The expandable support members 606, 608 may take any suitable shape and may be manufactured from any suitable materials. For example, in one embodiment, the expandable support members 606, 608 are each configured to expand from a stowed configuration (e.g., when compressed within the elongate flexible device 402, a sheath 610 as illustrated in FIG. 6B, or other housing used for delivering the electrode treatment system 600 to the target tissue site) to a deployed configuration (illustrated in FIG. 6A) upon their release from the elongate flexible device 402 or the sheath 610 as further described in detail below. In one embodiment, the expandable support members 606, 608 may be formed as braided mesh frames from any suitable material. For example, in one embodiment, the braided mesh frames may be formed from a metal alloy wire that is woven into a generally conical, hemispherical, semi-ellipsoidal, or other suitably-shaped structure having desired flexibility and radial strength characteristics. In some examples, the braided mesh frame is compressible within the sheath 610 (or the elongate flexible device 402) as illustrated in FIG. 6B and expandable upon release from the sheath 610 as illustrated in FIG. 6A. In one embodiment, the expandable support members 606, 608 may be formed from nickel titanium (e.g., nitinol) or from other suitable materials that preferably exhibit superelastic properties allowing the expandable support members 606, 608 to undergo large deformations (such as compressive forces within the sheath 610) and then return to an undeformed shape upon removal of the external forces (such as the expanded position illustrated in FIG. 6A).

As illustrated in FIG. 6A, the electrode treatment system 600 includes a first electrode 612 coupled to the first expandable support member 606 and a second electrode 614 coupled to the second expandable support member 608. The electrodes 612, 614 may include any suitable arrangement and may be coupled to the respective expandable support members 606, 608 in any suitable fashion. For example, in one embodiment, the electrodes 612, 614 may each be a ring-shaped structure disposed on a distal end portion 616, 618 of the respective expandable support members 606, 608. In other embodiments, the electrodes 612, 614 may be coupled to or otherwise incorporated along any region of the expandable support members 606, 608. In a similar fashion as described previously, the electrodes 612, 614 are configured for collectively delivering energy to a tissue surface for treatment. Electronic signals to the electrodes 612, 614 may be provided by way of the controller 14 and/or the control system 112 of FIGS. 1-2 as discussed previously.

In some embodiments, an exterior surface of the expandable support members 606, 608 (e.g., braided mesh frames) may be coated with a silicon or other suitable polymer material (or may be made from such material) to form a protective coating layer for reducing frictional forces and potential trauma during delivery, deployment, and retraction of the electrode treatment system 600. In addition, the protective coating layer may also help prevent the electrodes 612, 614 from sticking against the tissue during retraction of the electrodes 612, 614 after completion of treatment. In some embodiments, the electrodes 612, 614 may be coated with chemicals or other suitable materials to aid the electrolytic process and/or to prevent corrosion or oxidation of the electrodes 612, 614 during the electrolytic process.

As noted above, the electrode treatment system 600 may include a sheath 610 for housing the first flexible member 602 and the second flexible member 604 during delivery and retraction of the electrode treatment system 600. With particular reference to FIG. 6B, in one example arrangement, the sheath 610 includes a hollow interior with a cavity 620 extending therethrough and opening at a distal end portion 622 of the sheath 610. When the flexible members 602, 604 are positioned within the cavity 620 of the sheath 610, the first expandable support member 606 and the second expandable support member 608 are in a compressed, stowed configuration within the sheath 610. To deploy the electrodes 612, 614 for treatment, the first flexible member 602 and the second flexible member 604 are moved along an axial direction (e.g., translated along a longitudinal axis B) through the cavity 620 and distally of the distal end portion 622 of the sheath 610. As the flexible members 602, 604 move distally out of the sheath 610, the first expandable support member 606 and the second expandable support member 608 radially expand outwardly from the stowed configuration to the deployed configuration. As illustrated in FIG. 6A, the first flexible member 602 and the second flexible member 604 each extend through the cavity 620 and distally relative to the distal end portion 622 of the sheath 610 when the electrode treatment system 600 is in a deployed configuration for treatment. In the deployed configuration, the expandable support members 606, 608 are radially expanded outwardly relative to Axis B to deploy the electrodes 612, 614. Upon completion of treatment, the first flexible member 602 and the second flexible member 604 may be retracted backward (e.g., proximally) along the axis B into the cavity 620 to retract the first expandable support member 606 and the second expandable support member 608 from the deployed configuration back to the stowed configuration.

In some embodiments, the first expandable support member 606 and the second expandable support member 608 each expand without additional application of force (e.g., passive or resilient expansion) from the stowed configuration to the deployed configuration in response to the respective first flexible member 602 and the second flexible member 604 extending distally of the sheath 610 as described above. For example, once the flexible members 602, 604 are driven distally of the sheath 610, the expandable support members 606, 608 automatically expand to the deployed configuration. In some embodiments, this expansion behavior may result from the material selection (e.g., nitinol or other superelastic material) of the expandable support members 606, 608. In other embodiments, the expandable support member may be actively expanded (e.g., upon application of additional force). For example, one or more expansion members (e.g., an expandable balloon or other suitable member) may be coupled to one or both of the expandable support members 606, 608, where the expansion member is radially expandable to urge expansion of the expandable support members 606, 608 from the stowed configuration to the deployed configuration. Conversely, the expansion member can also urge retraction of the expandable support member from the deployed configuration to the stowed configuration for retraction or removal of the electrode treatment systems described herein.

As mentioned previously, in some embodiments, a position of the first expandable support member 606 is adjustable independently of a position of the second expandable support member 608 to adjust a target spacing between the first electrode 612 and the second electrode 614 for treatment of the tissue surface. For example, one or both of the first flexible members 602 and the second flexible member 604 may be driven within the sheath 610 and relative to each other to arrange the electrodes 612, 614 at a target spacing as desired. In some example embodiments, the position and spacing of the electrodes 612, 614 may be adjusted manually. In other embodiments, the electrode treatment system 600 may be in operable communication with a control system for operation. Additional details of some example embodiments are provided below.

With collective reference to FIGS. 1-6B, in one embodiment system, a control system 112 (or controller 14) is in operable communication with the electrode treatment system 600 and may be used to perform the adjustment actions and manipulation of the flexible members 602, 604 described above. For example, the control system 112 may be operable to adjust a relative position of the expandable support members 606, 608 by driving one or both of the flexible members 602, 604 (to which the respective expandable support member 606, 608 are coupled) relative to one another. It should be understood that any actions relating to adjustment of the flexible members 602, 604 and the components coupled thereto (such as the expandable support members 606, 608 and the electrodes 612, 614) described above with reference to FIGS. 4-6B may also be completed by way of, and/or assisted by, the control system 112.

In some embodiments, the control system 112 may be operable to independently adjust a relative position of the expandable support members 606, 608 to modulate the spacing between the electrodes 612, 614 based on a target energy level for the applied electric field for achieving a desired depth of penetration for application to the target tissue surface. Once the target spacing has been set as desired, the control system may induce a voltage based on the target energy level and the target spacing of the electrodes 612, 614 to treat the tissue surface. In some embodiments, the control system 112 may be operable to vary the applied energy level as needed based on the spacing of the electrodes 612, 614 (and/or vice versa). In other embodiments, the control system 112 may modulate the spacing between the electrodes 612, 614 as needed based on the desired strength of the applied energy level.

In some embodiments, the control system 112 may further be in operable communication with a locking system 118 (see FIG. 2). In such configurations, the locking system 118 is in operable communication with the flexible members 602, 604 and may be operable to selectively lock a position of the flexible members 602, 604 (and by extension, lock the position of the expandable support members 606, 608 and the electrodes 612, 614). Locking the position of these components helps ensure that the target spacing between the first electrode 612 and the second electrode 614 is maintained during treatment of the tissue surface. For example, in one embodiment, the control system 112 may be used to adjust a position of the flexible members 602, 604 and ensure the spacing between the electrodes 612, 614 is as desired for treatment. Once the spacing is set as desired, the control system 112 may communicate with the locking system 118 to lock the position of the flexible members 602, 604 and prevent further adjustments or movement. With the position locked, the control system 112 may thereafter apply the target energy level to the electrodes 612, 614 for treatment.

In I some embodiments, after treatment at a first target tissue site is completed, the control system 112 may advance or retract the electrode treatment system 600 to a second target tissue site, which may be adjacent the first target tissue site. This further adjustment may be completed while the flexible members 602, 604 (and the expandable support members 606, 608 and the electrodes 612, 614) remain in the locked position for subsequent treatment to maintain consistent energy application to the tissue at the two tissue sites. This repositioning process of the electrode treatment system 600 may be repeated as needed to complete treatment of larger sections of tissue. In other embodiments, the control system 112 is further operable to adjust a rate of movement of the flexible members 602, 604 (and the expandable support members 606, 608 and the electrodes 612, 614) and move the flexible members 602, 604 at the desired rate of movement while inducing the voltage for continuous treatment of the tissue surface. In yet further embodiments, spacing between the electrodes or expandable support members is adjusted such that it is different between a first target tissue site and a second target tissue site.

As described above, in some embodiments, expansion members (not shown), such as inflatable balloons and the like, may be coupled to the expandable support members 606, 608 to aid in their radial expansion to the deployed configuration from the stowed configuration. In such embodiments, the control system 112 may be in communication with the expansion members and operable to control an expansion rate of the one or more expansion members to ensure the expandable support members 606, 608 are expanded at a desired rate.

In the electrode treatment system 600 of FIGS. 6A, 6B, the electrodes 612, 614 are illustrated and described as coupled to the respective distal portions 616, 618 of the expandable support members 606, 608. In other embodiments, the expandable support members 606, 608 may be made of suitable conductive material such that the expandable support members 606, 608 act as the electrodes themselves (or at least the portions of the expandable support members 606, 608 that contact the tissue act as electrodes when the expandable support members 606, 608 are expanded). In other embodiments, a combination of these features may be achieved such that the electrodes 612, 614 along the distal portions 616, 618 may be selectively energized and used for electroporation, and the expandable support members 606, 608 (e.g., the portions contacting the tissue) may be selectively energized and used for electrolysis, where the larger surface area of the expandable support members 606, 608 helps create a larger volume of electrolytics products to optimize treatment.

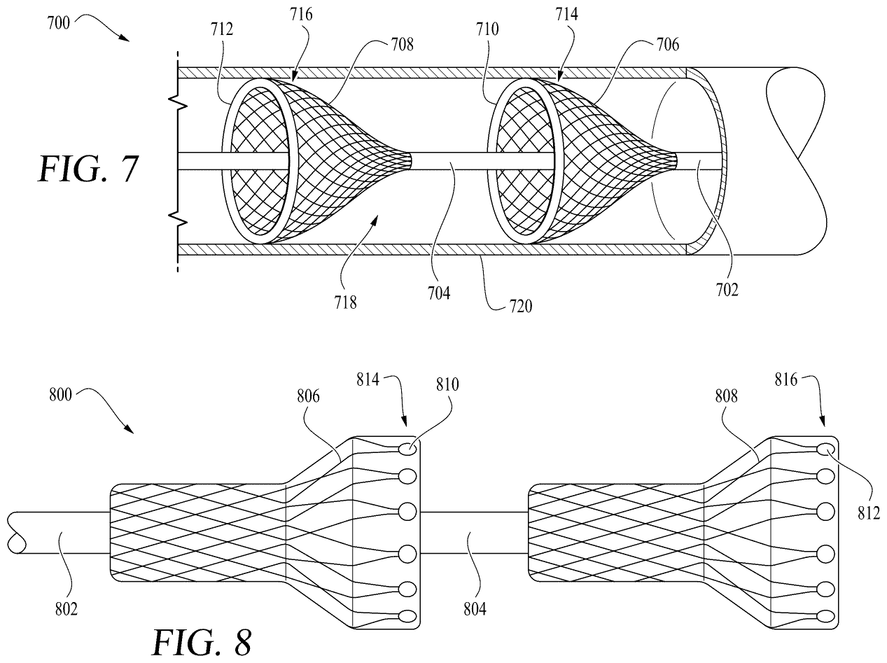

FIG. 7 illustrates another example of an electrode treatment system 700 for delivering electrodes 710, 712 to a target region for treatment in accordance with examples described herein. Generally, the electrode treatment system 700 may have the same or substantially similar features operating in a similar fashion as the corresponding components of the electrode treatment systems 400, 600 described previously with general reference to FIGS. 4-6B. Accordingly, certain details of the electrode treatment system 700 may not be further described in detail below to avoid repetition and/or obscuring more pertinent details of the embodiment. It should be understood that the description of corresponding components and their related functionality as described with reference to electrode treatment systems 400, 600 may apply equally to the electrode treatment system 700.

Briefly, with reference to FIG. 7, the electrode treatment system 700 includes a first flexible member 702 and a second flexible member 704, each of which include an expandable support member 706, 708, respectively. Each expandable support member 706, 708 includes an electrode 710, 712 coupled along a respective distal end portion 714, 716 of the expandable support members 706, 708. In FIG. 7, the electrodes 710, 712 are illustrated as substantially circular or ring-shaped electrodes as described previously but may take other suitable configurations in other embodiments. The electrode treatment system 700 may include expandable support members 706, 708 comprising braided mesh stents with a generally conical profile (or other suitable profiles as noted previously) when in the deployed configuration. When the expandable support members 706, 708 are deployed within a lumen 718, the electrodes 710, 712 radially expand outwardly and are positioned adjacent (or in contact with) the tissue 720 for treatment. Thereafter, the electrodes 710, 712 may be energized via the control system 112 in a similar fashion as described previously.

FIG. 8 illustrates another example of an electrode treatment system 800 for delivering electrodes 810, 812 to a target region for treatment in accordance with examples described herein. Generally, the electrode treatment system 800 may have the same or substantially similar features operating in a similar fashion as the corresponding components of the electrode treatment systems 400, 600 described previously with general reference to FIGS. 4-6B. Accordingly, certain details of the electrode treatment system 800 may not be further described in detail below to avoid repetition and/or obscuring more pertinent details of the embodiment. It should be understood that the description of corresponding components and their related functionality as described with reference to electrode treatment systems 400, 600 may apply, in whole or in part, to the electrode treatment system 800.

Briefly, with reference to FIG. 8, the electrode treatment system 800 includes a first flexible member 802 and a second flexible member 804, each of which include an expandable support member 806, 808, respectively. Each expandable support member 806, 808 includes an electrode 810, 812 coupled along a respective distal end portion 814, 816 of the expandable support members 806, 808. In one embodiment, the electrodes 810, 812 may include a plurality of point-contact or marker-like electrodes disposed on the distal end portions 814, 816 of the expandable support members 806, 808. The electrode treatment system 800 may include expandable support members 806, 808 comprising braided mesh stents with any suitable profile when in the deployed configuration. In a similar fashion as described previously, when the expandable support members 806, 808 are deployed within a lumen (e.g., lumen 718 of FIG. 7) the electrodes 810, 812 radially expand outwardly and are positioned adjacent (or in contact with) the tissue (e.g., tissue 720 of FIG. 7) for treatment. Thereafter, the electrodes 810, 812 may be energized via the control system 112 in a similar fashion as described previously.

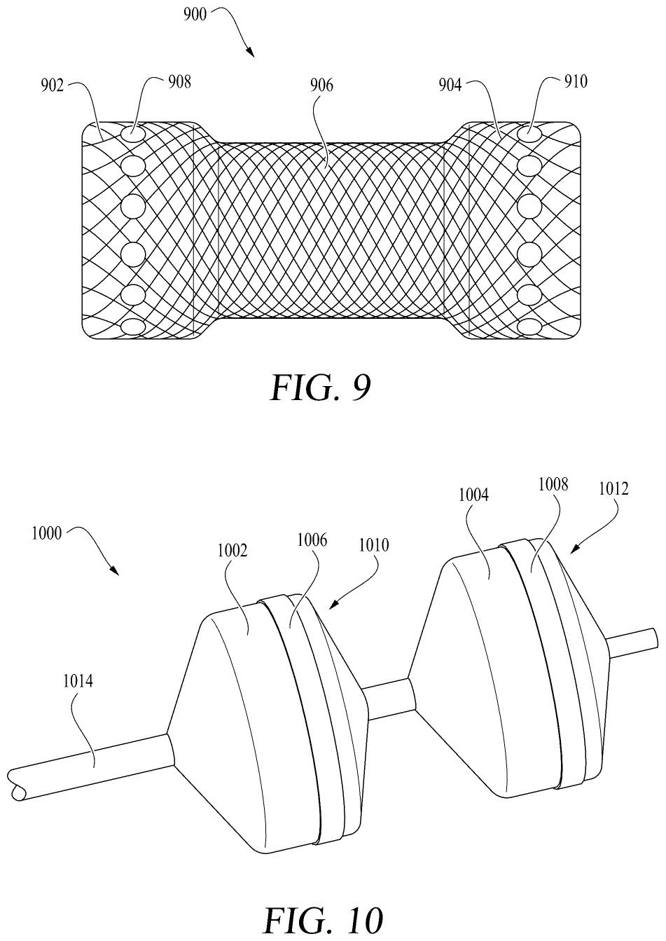

FIG. 9 illustrates another example of an electrode treatment system 900 for delivering electrodes 908, 910 to a target region for treatment in accordance with examples described herein. With reference to FIG. 9, the electrode treatment system 900 includes a first expandable support member 902 and a second expandable support member 904, each of which have an electrode 908, 910 coupled thereto. In one embodiment, the electrodes 908, 910 may be point-contact or marker-like electrodes as illustrated but may take any other suitable forms in other embodiments, including ring-shaped electrodes similar to those described previously with reference to FIGS. 6A, 6B, and 7. The electrode treatment system 900 further includes a frame 906 extending between and coupling the expandable support members 902, 904. In some examples, the frame 906 is expandable and compressible to adjust the spacing between the expandable support members 902, 904 (and the electrodes 908, 910 by extension) relative to one another. In one example, the frame 906, the first expandable support member 902, and the second expandable support member 904 may each be braided mesh frames. The braided mesh frames may be made of any suitable material, such as nitinol, or other material that preferably exhibits superelastic material properties to facilitate expansion and contraction of the electrode treatment system 900 as desired. In some embodiments, the electrode treatment system 900 may further include one or more expansion members (e.g., a balloon or the like) that are radially expandable to expand the frame 906 and adjust the spacing of the electrodes 908, 910 relative to one another.

In some embodiments, an exterior surface of the expandable support members 902, 904 and the frame 906 may be coated with a silicon or other suitable polymer material (or may be made from such material) to create a protective coating layer to reduce frictional forces and potential trauma during delivery, deployment, and retraction of the electrode treatment system 900. In addition, the protective coating layer may also help prevent the electrodes 908, 910 from sticking against the tissue after treatment and during the process of retracting and removing the electrode treatment system 900.

In some examples, the electrode treatment system 900 is deployed and used in a substantially similar manner as described previously with reference to other embodiments. For example, the electrode treatment system 900 may be initially compressed within a sheath (e.g., sheath 610 of FIG. 6A) or within the body of an elongate flexible device (e.g., elongate flexible device 202 of FIG. 2 and elongate flexible device 402 of FIG. 4) or other housing used for delivering the electrode treatment system 900 to the target tissue site. In this stowed and compressed configuration, the electrode treatment system 900 is delivered to the target tissue site for treatment. At the tissue site, the electrode treatment system 900 may be advanced out of the sheath 610 (or other suitable housing) to position the electrodes 908, 910 adjacent or in contact with the tissue for treatment in a similar fashion as described previously. In some embodiments, the expandable support members 902, 904 and the frame 906 automatically expand upon advancement out of the sheath 610 due to their material properties, such as when using nitinol or other superelastic material to form the expandable support members 902, 904 and the frame 906. In other embodiments, expansion members (e.g., inflatable balloons or other suitable devices) may be used to urge and support expansion of these components as described previously. After treatment is completed, the electrode treatment system 900 may be advanced or retracted to treat a different tissue site or may be retracted back into the sheath 610 (or other housing) for removal from the patient. In a similar fashion as described with reference to previous examples, upon retraction into the sheath 610, the expandable support members 902, 904 and the frame 906 are compressed to a stowed configuration to maintain a low overall profile for the electrode treatment system 900 and facilitate removal.

FIG. 10 illustrates another example of an electrode treatment system 1000 for delivering electrodes 1006, 1008 to a target region for treatment in accordance with examples described herein. Generally, the electrode treatment system 1000 may have the same or substantially similar features as the electrode treatment systems 400, 600 described previously with reference to FIGS. 4-6B. Accordingly, certain details of the electrode treatment system 1000 may not be further described in detail below to avoid repetition and/or obscuring more pertinent details of the embodiment. It should be understood that the description of corresponding components and their related functionality as described with reference to electrode treatment systems 400, 600 may apply equally to the electrode treatment system 1000. Briefly, with reference to FIG. 10, the electrode treatment system 1000 includes a first expandable support member 1002 and a second expandable support member 1004, each of which have an electrode 1006, 1008 coupled thereto. In one embodiment, the electrodes 1006, 1008 may be ring-shaped electrodes similar to those described previously with reference to FIGS. 6A, 6B, and 7 but may take any other suitable forms in other embodiments, including point-contact or marker-like electrodes described with reference to FIGS. 8 and 9, or other suitable electrodes. In one embodiment, the electrodes 1006, 1008 are disposed on or adjacent to the distal end portions 1010, 1012 of the expandable support member 1002, 1004.

With reference to FIG. 10, in some embodiments, the expandable support members 1002, 1004 may each be expansion members that are radially expandable relative to a flexible member 1014. The expansion members may be expanded in a variety of different manners. For example, in some embodiments, the expansion members may be inflated with a fluid, such as air, saline, a radiopaque solution, and the like. In some embodiments, the fluid may be introduced to the expansion members through a lumen (not shown) of the flexible member 1014. The expansion members may have a length of approximately 4 cm in an unexpanded configuration and may inflate to a diameter between 1 cm and 4 cm. In other embodiments, the length and inflatable diameter of the expansion members may vary depending on the characteristics and dimensions of the target site for tissue treatment. In other examples, the expansion members may be expanded in other suitable ways (e.g., rolling, unfurling, pushing). In the illustrated embodiment, the expansion members are illustrated as inflatable balloons. In some embodiments, the balloon may be a compliant balloon made of polyurethane, silicone, or other suitable material designed to inflate several times its nominal size. In other embodiments, the balloon may be a semi-compliant balloon made of polyurethane or other suitable material designed for less expansion as compared to a compliant balloon.

In some embodiments, the expansion member may be housed within a sheath (e.g., sheath 610 of FIG. 6A) or other suitable housing and configured to extend therethrough for deployment in a similar fashion as described previously. Briefly, once the electrode treatment system 1000 is advanced to target tissue site, the expandable support members 1002, 1004 may be advanced distally of the sheath 610 and inflated (or otherwise expanded) to expand the expandable support members 1002, 1004 toward the target tissue at the treatment site. As the expandable support members 1002, 1004 are inflated, the electrodes 1006, 1008 are moved into position adjacent (or in contact with) the target tissue. In some embodiments, a rate and/or degree of expansion of the expandable support members 1002, 1004 may be controlled (such as via the control system 112) to ensure that the electrodes 1006, 1008 appropriately contact (or are otherwise positioned with sufficient proximity) for treatment of the tissue while avoiding over-expansion of the expandable support members 1002, 1004 that may result in tissue damage. With the electrodes 1006, 1008 in proper position relative to the tissue, the control system 112 may energize the electrodes 1006, 1008 to generate an electric field for treatment.

In some examples as described previously, the electrode treatment system 1000 may be advanced or retracted to different tissue sites to continue treatment as needed. In some embodiments, the expandable support members 1002, 1004 may be partially or entirely deflated (e.g., retracted, reduced) to facilitate movement of the electrode treatment system 1000 to the second (and other ensuing) tissue treatment site. Deflating the expandable support members 1002, 1004 prior to moving the electrode treatment system 1000 may facilitate repositioning of the electrode treatment system 1000 by decreasing its overall profile and may also reduce the risk of inadvertent injury caused by the electrode treatment system 1000 to surrounding tissue during movement. Upon completion of treatment, the expandable support members 1002, 1004 may be deflated and retracted back into the sheath 610 or other housing. Thereafter, the electrode treatment system 1000 may be removed.

The example embodiments of the various electrode treatment systems described with reference to the figures relate to a design where the target spacing between the electrodes is adjustable to improve flexibility and maximize overall treatment options. In other embodiments, the spacing between the electrodes may instead be fixed in the axial or longitudinal direction to ensure the electrodes are essentially spaced a set distance apart at all times. In this design, the electrode treatment system may be usable between various patients to treat any suitable tissue site without needed to adjust energy levels or otherwise compensate for changes in spacing between the electrodes. While having a fixed design for the electrodes may result in a shorter treatment length (e.g., a smaller treatable tissue region per deployment of the electrode treatment system) since the electrodes would likely be relatively close to one another to ensure the electrode treatment system may be deployed across a range of tissues (including tissues that may have a relatively narrow lumen), this potential tradeoff may be offset by regularly adjusting a position of the electrode treatment system during energy delivery at a controlled rate to collectively ablate larger tissue sections.

It should be understood that example embodiments provided herein of both the design of the electrode treatment systems and the potential clinical applications associated therewith are not intended to be limiting. Many other configurations of the instrument systems exist, as well as applications that would benefit from the use of the disclosed subject matter. In addition, it is to be appreciated that any one of the above embodiments or processes, or specific features associated therewith, may be combined with one or more other embodiments and/or processes or be separated and/or performed amongst separate devices or device portions in accordance with the present systems, devices, and methods.

Finally, the disclosure is intended to be merely illustrative of the present devices, apparatuses, systems, and methods and should not be construed as limiting the appended claims to any particular embodiment or group of embodiments. Thus, while the present disclosure has been described in particular detail with reference to exemplary embodiments, it should also be appreciated that numerous modifications and alternative embodiments may be practiced without departing from the broader and intended spirit and scope of the present disclosure as set forth in the claims that follow. Accordingly, the specification and drawings are to be regarded in an illustrative manner and are not intended to limit the scope of the appended claims.

1. An electrode treatment system, the system comprising:
a first flexible member with a first expandable support member coupled thereto, wherein the first expandable support member is configured to expand from a stowed configuration to a deployed configuration;
a second flexible member with a second expandable support member coupled thereto, wherein the second expandable support member is configured to expand from a stowed configuration to a deployed configuration; and
a first electrode coupled to the first expandable support member and a second electrode coupled to the second expandable support member, wherein the first electrode and the second electrode are configured for collectively delivering energy to a tissue surface for treatment thereof,
wherein a position of the first expandable support member is adjustable independently of a position of the second expandable support member to adjust a target spacing between the first electrode and the second electrode for treatment of the tissue surface.

2. The system of claim 1, wherein the first flexible member includes a hollow interior with a cavity extending therethrough and opening at a distal end portion of the first flexible member, and wherein the second flexible member extends through the cavity and distally of the distal end portion of the first flexible member.

3. The system of claim 1, further comprising a sheath including a hollow interior with a cavity extending therethrough and opening at a distal end portion of the sheath, and wherein both the first flexible member and the second flexible member each extend through the cavity and distally relative to the distal end portion of the sheath.

4. The system of claim 3, wherein the first expandable support member and the second expandable support member are in the stowed configuration within the sheath when the first flexible member and the second flexible member are positioned within the cavity of the sheath.

5. The system of claim 3, wherein the first flexible member and the second flexible member are movable along an axial direction through the cavity and distally of the sheath to expand the first expandable support member and the second expandable support member from the stowed configuration to the deployed configuration, and wherein the first flexible member and the second flexible member are retractable along the axial direction into the cavity to retract the first expandable support member and the second expandable support member from the deployed configuration to the stowed configuration.

6. The system of claim 5, wherein the first expandable support member and the second expandable support member each spontaneously expand from the stowed configuration to the deployed configuration in response to the respective first flexible member and the second flexible member extending distally of the sheath.

7. The system of claim 5, further comprising one or more expansion members coupled to one or both of the first expandable support member and the second expandable support member, wherein the one or more expansion members are radially expandable to expand the one or both of the first expandable support member and the second expandable support member from the stowed configuration to the deployed configuration.

8. The system of claim 1, wherein the first expandable support member and the second expandable support member each include a braided mesh frame.

9. The system of claim 8, wherein an exterior surface of the braided mesh frame includes a protective coating to reduce tissue trauma during delivery to the tissue surface.

10. The system of claim 1, wherein the first expandable support member and the second expandable support member are each expansion members that are radially expandable.

11. The system of claim 1, wherein the first electrode is a ring-shaped structure and is disposed on a distal end portion of the first expandable support member, and wherein the second electrode is a ring-shaped structure and is disposed on a distal end portion of the second expandable support member.

12. The system of claim 1, wherein the first electrode includes a plurality of point-contact electrodes disposed on a distal end portion of the first expandable support member, and wherein the second electrode includes a plurality of point-contact electrodes disposed on a distal end portion of the second expandable support member.

13. The system of claim 1, further comprising a locking mechanism operable to selectively lock the position of the first expandable support member relative to the position of the second expandable support member to maintain the target spacing between the first electrode and the second electrode during treatment of the tissue surface.

14. The system of claim 1, wherein the system further includes a frame extending between and coupling the first expandable support member and the second expandable support member with one another, the frame being adjustable to adjust the target spacing between the first electrode and the second electrode.

15. The system of claim 14, wherein the frame, the first expandable support member, and the second expandable support member are each braided mesh frames.

16. The system of claim 14, further comprising at least one expansion member coupled to the frame, wherein the expansion member is radially expandable to expand the frame and adjust the target spacing of the first electrode and the second electrode.

17. An electrode treatment system, the system comprising:
a delivery system including a first expandable support member and a second expandable support member, wherein the first expandable support member includes a first electrode coupled thereto and the second expandable support member includes a second electrode coupled thereto, wherein the first electrode and the second electrode are configured for delivering energy to a tissue surface for treatment thereof; and
a controller in operable communication with the delivery system, the controller configured to:
independently adjust a relative position of the first expandable support member and the second expandable support member based on a target energy level for application to the tissue surface to adjust a target spacing between the first electrode and the second electrode for treatment of the tissue surface; and
induce a voltage based on the target energy level and the target spacing of the first electrode and the second electrode to treat the tissue surface.

18. The system of claim 17, wherein the first expandable support member is coupled to a first flexible member and the second expandable support member is coupled to a second flexible member, and wherein the controller is operable to drive one or both of the first flexible member and the second flexible member relative to one another.

19. The system of claim 18, wherein the first flexible member includes a hollow interior with a cavity extending therethrough and opening at a distal end portion of the first flexible member, and wherein the second flexible member is adjustable via the controller through the cavity and distally of a distal end portion of the first flexible member.

20. The system of claim 18, further comprising a sheath including a hollow interior with a cavity extending therethrough and opening at a distal end portion of the sheath, and wherein the first flexible member and the second flexible member are each adjustable via the controller through the cavity of the sheath.

21. The system of claim 18, wherein the first flexible member and the second flexible member are movable along an axial direction through a cavity of a sheath and distally of the sheath to expand the first expandable support member and the second expandable support member from a stowed configuration to a deployed configuration, and wherein the first flexible member and the second flexible member are retractable along the axial direction into the cavity to retract the first expandable support member and the second expandable support member from the deployed configuration to the stowed configuration.

22. The system of claim 17, further comprising a locking mechanism in operable communication with the controller, wherein the locking mechanism is operable to selectively lock the position of the first expandable support member relative to the position of the second expandable support member via the controller to maintain the target spacing between the first electrode and the second electrode during treatment of the tissue surface.

23. The system of claim 22, wherein when the first electrode and the second electrode are locked in the target spacing, the controller is further operable to adjust a rate of movement of the first expandable support member and the second expandable support member while inducing the voltage for continuous treatment of the tissue surface.

24. The system of claim 17, further comprising one or more expansion members coupled to one or both of the first expandable support member and the second expandable support member, wherein the one or more expansion members are radially expandable to expand the one or both of the first expandable support member and the second expandable support member and adjust the target spacing between the first electrode and the second electrode.

25. The system of claim 24, wherein the controller is in communication with the one or more expansion members, and wherein the controller is operable to control an expansion rate of the one or more expansion members.

26-44. (canceled)
